(19) United States
(12) Patent Application Publication
(10) Pub. No.: US 2025/0138180 A1
(43) Pub. Date: May 1, 2025

(54) SYSTEM AND METHOD FOR MONITORING PLUGGING OF BASKET ASSEMBLIES OF AN AGRICULTURAL IMPLEMENT

(71) Applicant: CNH Industrial America LLC, New Holland, PA (US)

(72) Inventors: Brittany Schroeder, Bunker Hill, IN (US); Joshua D. Knoblauch, Lowpoint, IL (US)

(21) Appl. No.: 18/494,929

(22) Filed: Oct. 26, 2023

(51) Int. Cl. G01S 13/88; A01B 29/04; A01B 79/00; G01N 33/24

(52) U.S. Cl. CPC G01S 13/88; A01B 29/048; A01B 79/00; G01N 33/24; G01N 33/245

(57) ABSTRACT

An agricultural system for monitoring plugging of basket assemblies of an agricultural implement includes a plugging sensor positioned within an interior of a monitored basket assembly, where the plugging sensor is configured to transmit detection signals at different frequencies across a field of detection along the interior of the basket assembly and along the rotational axis, and generate data indicative of return signals received based on reflection of the detection signals at the different frequencies off at least one surface. Additionally, the agricultural system includes a computing system configured to receive the data generated by the plugging sensor and determine when the basket assembly is experiencing a plugged condition based at least in part on the data generated by the plugging sensor.

### FIELD OF THE INVENTION

The present disclosure generally relates to agricultural implements and, more particularly, to systems and methods for monitoring plugging of ground-engaging tools of an agricultural implement, such as rolling basket assemblies, based at least in part on reflection of different frequencies.

### BACKGROUND OF THE INVENTION

It is well known that, to attain the best agricultural performance from a field, a farmer must cultivate the soil, typically through a tillage operation. Modern farmers perform tillage operations by pulling a tillage implement behind an agricultural work vehicle, such as a tractor. Tillage implements typically include one or more ground-engaging tools configured to engage the soil as the implement is moved across the field. For example, in certain configurations, the implement may include one or more harrow disks, leveling disks, rolling baskets, shanks, tines, and/or the like. Such ground-engaging tool(s) loosen and/or otherwise agitate the soil to prepare the field for subsequent planting operations.

During tillage operations, field materials, such as residue, soil, rocks, mud, and/or the like, may become trapped or otherwise accumulate on and/or within ground-engaging tools or between adjacent ground-engaging tools. For instance, material accumulation will often occur around the exterior of a basket assembly (e.g., on the blades or bars of the basket assembly) and/or within the interior of the basket assembly, within the curvature of shanks, between ganged disks, and/or the like. Such accumulation of field materials may prevent the basket assembly from performing in a desired manner during the performance of a tillage operation. In such instances, it is often necessary for the operator to take certain corrective actions to remove the material accumulation. However, it is typically difficult for the operator to detect or determine a plugged condition of a basket assembly or any other suitable ground-engaging tool(s) when viewing the tools from the operator's cab.

Accordingly, an improved system and method for monitoring plugging of ground-engaging tools, particularly rolling basket assemblies, of an agricultural implement would be welcomed in the technology.

### BRIEF DESCRIPTION OF THE INVENTION

Aspects and advantages of the invention will be set forth in part in the following description, or may be obvious from the description, or may be learned through practice of the invention.

In one aspect, the present subject matter is directed to an agricultural system for monitoring plugging of basket assemblies of an agricultural implement. The agricultural system may include a basket assembly configured to be supported by a frame of an agricultural implement for rotation relative to the frame about a rotational axis, where the basket assembly may extend across a lateral width defined between a first lateral end and a second lateral end spaced apart along the rotational axis. The agricultural system may further include a plugging sensor positioned within an interior of the basket assembly. The plugging sensor may particularly be configured to transmit detection signals at different frequencies across a field of detection along the interior of the basket assembly and along the rotational axis and may be configured to generate data indicative of return signals received based on reflection of the detection signals at the different frequencies off at least one surface. Additionally, the agricultural system may include a computing system configured to receive the data generated by the plugging sensor and determine when the basket assembly is experiencing a plugged condition based at least in part on the data generated by the plugging sensor.

In another aspect, the present subject matter is directed to an agricultural method for monitoring plugging of a basket assembly of an agricultural implement, where the basket assembly may be configured to be supported by a frame of the agricultural implement for rotation relative to the frame about a rotational axis, and where the basket assembly may extend across a lateral width defined between a first lateral end and a second lateral end spaced apart along the rotational axis. The agricultural method may include receiving, with a computing system, data generated by a plugging sensor positioned within an interior of the basket assembly, with the plugging sensor being configured to transmit detection signals at different frequencies across a field of detection along the interior of the basket assembly and along the rotational axis, and with the data being indicative of return signals received by the plugging sensor based on reflection of the detection signals at the different frequencies off at least one surface. The agricultural method may further include determining, with the computing system, when the basket assembly is experiencing a plugged condition based at least in part on the data generated by the plugging sensor. Additionally, the agricultural method may include performing, with the computing system, a control action associated with the agricultural implement when the basket assembly is experiencing the plugged condition.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of one embodiment of an agricultural implement coupled to a work vehicle in accordance with aspects of the present subject matter;

FIG. 2 illustrates another perspective view of the agricultural implement shown in FIG. 1 in accordance with aspects of the present subject matter;

FIG. 3 illustrates a partial perspective view of finishing tools positioned at an aft end of the implement shown in FIGS. 1 and 2 in accordance with aspects of the present subject matter;

FIG. 4 illustrates a schematic view of one embodiment of a system for monitoring plugging of basket assemblies of an agricultural implement in accordance with aspects of the present subject matter; and

FIG. 5 illustrates a flow diagram of one embodiment of a method for monitoring plugging of basket assemblies of an agricultural implement in accordance with aspects of the present subject matter.

Repeat use of reference characters in the present specification and drawings is intended to represent the same or analogous features or elements of the present technology.

### DETAILED DESCRIPTION OF THE INVENTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a further embodiment. Thus, it is intended that the present invention covers such modifications and variations as come within the scope of the appended claims and their equivalents.

In this document, relational terms, such as first and second, top and bottom, and the like, are used solely to distinguish one entity or action from another entity or action, without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

As used herein, the terms “first,” “second,” and “third” may be used interchangeably to distinguish one component from another and are not intended to signify a location or importance of the individual components. The terms “coupled,” “fixed,” “attached to,” and the like refer to both direct coupling, fixing, or attaching, as well as indirect coupling, fixing, or attaching through one or more intermediate components or features, unless otherwise specified herein. The term “selectively” refers to a component's ability to operate in various states (e.g., an ON state and an OFF state) based on manual and/or automatic control of the component.

Furthermore, any arrangement of components to achieve the same functionality is effectively “associated” such that the functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected” or “operably coupled” to each other to achieve the desired functionality, and any two components capable of being so associated can also be viewed as being “operably couplable” to each other to achieve the desired functionality. Some examples of operably couplable include, but are not limited to, physically mateable, physically interacting components, wirelessly interactable, wirelessly interacting components, logically interacting, and/or logically interactable components.

The singular forms “a,” “an,” and “the” include plural references unless the context clearly dictates otherwise.

Approximating language, as used herein throughout the specification and claims, is applied to modify any quantitative representation that could permissibly vary without resulting in a change in the basic function to which it is related. Accordingly, a value modified by a term or terms, such as “about,” “approximately,” “generally,” and “substantially,” is not to be limited to the precise value specified. In at least some instances, the approximating language may correspond to the precision of an instrument for measuring the value, or the precision of the methods or apparatus for constructing or manufacturing the components and/or systems. For example, the approximating language may refer to being within a ten percent margin.

Moreover, the technology of the present application will be described in relation to exemplary embodiments. The word “exemplary” is used herein to mean “serving as an example, instance, or illustration.” Any embodiment described herein as “exemplary” is not necessarily to be construed as preferred or advantageous over other embodiments. Additionally, unless specifically identified otherwise, all embodiments described herein will be considered exemplary.

As used herein, the term “and/or,” when used in a list of two or more items, means that any one of the listed items can be employed by itself, or any combination of two or more of the listed items can be employed. For example, if a composition or assembly is described as containing components A, B, and/or C, the composition or assembly can contain A alone; B alone; C alone; A and B in combination; A and C in combination; B and C in combination; or A, B, and C in combination.

In general, the present subject matter is directed to systems and methods for monitoring plugging of basket assemblies of agricultural implements. Specifically, in several embodiments, the disclosed system may include one or more plugging sensors positioned within an interior of a given basket assembly. For instance, a plugging sensor may be positioned at a lateral end of the basket assembly such that a field of view of the plugging sensor is directed towards the other lateral end of the basket assembly. Each plugging sensor (e.g., multi-frequency ground penetrating radar, multi-frequency LIDAR, and/or the like) is configured to transmit detection signals at different frequencies across a field of detection along the interior of the basket assembly, generally along a rotational axis of the basket assembly, and to generate data indicative of return signals received based on reflection of the detection signals at the different frequencies off at least one surface. For instance, each frequency may generally be associated with a different distance along the rotational axis and/or may have a respective baseline return signal profile when the basket assembly is unplugged. As the basket assembly becomes plugged, a return signal profile of the return signal reflected from transmitted detection signals at the different frequencies may change (e.g., from the respective baseline return signal profile). As such, a computing system may determine when the basket assembly is experiencing the plugged condition based at least in part on the data generated by the plugging sensors. Once it is determined that the ground-engaging tool is experiencing a plugged condition, an appropriate control action may then be executed, such as by notifying the operator of the plugged condition or by performing an automated control action. As such, the disclosed system may generally reduce inspection time and increase productivity, while also preventing the basket assemblies from being operated when plugged.

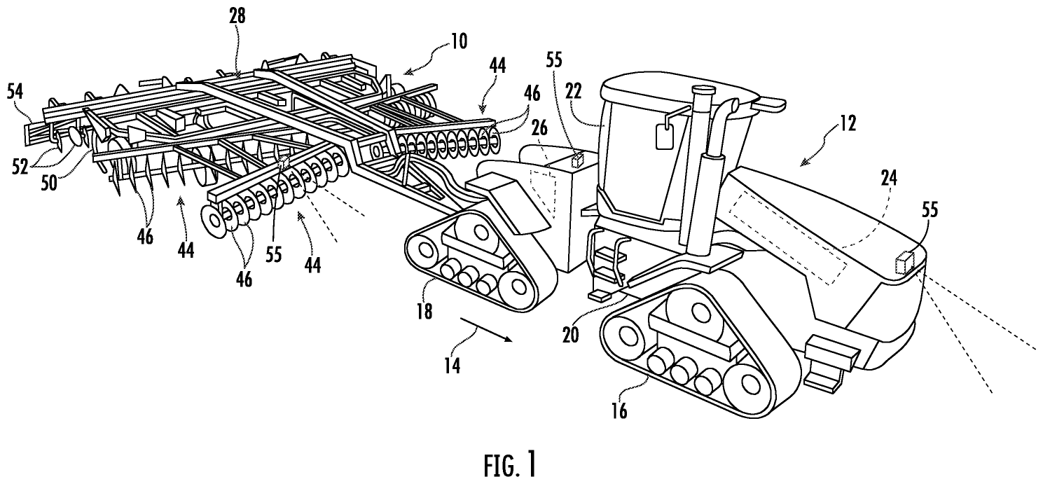

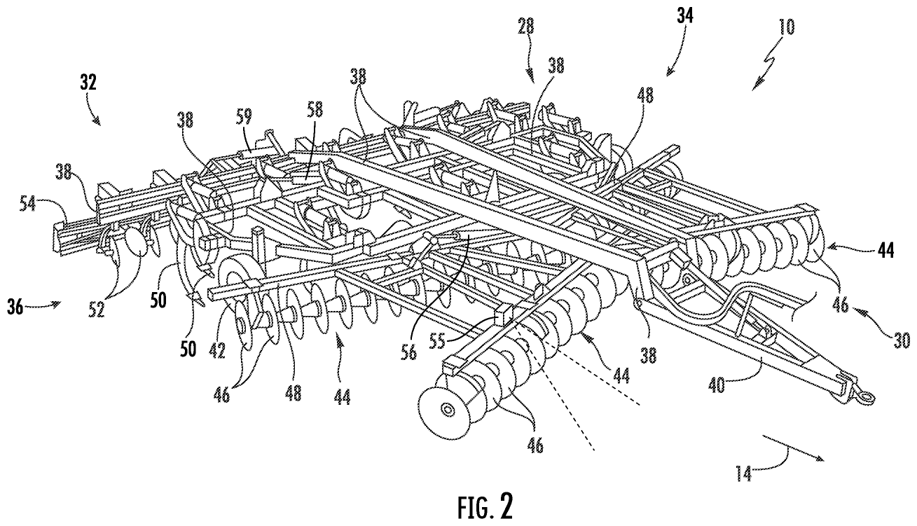

Referring now to the drawings, FIGS. 1 and 2 illustrate differing perspective views of one embodiment of an agricultural implement 10 in accordance with aspects of the present subject matter. Specifically, FIG. 1 illustrates a perspective view of the agricultural implement 10 coupled to a work vehicle 12. Additionally, FIG. 2 illustrates a perspective view of the implement 10, particularly illustrating various components of the implement 10.

In general, the implement 10 may be configured to be towed across a field in a direction of travel (e.g., as indicated by arrow 14 in FIG. 1) by the work vehicle 12. As shown, the implement 10 may be configured as a tillage implement, and the work vehicle 12 may be configured as an agricultural tractor. However, in other embodiments, the implement 10 may be configured as any other suitable type of implement, such as a seed-planting implement, a fertilizer-dispensing implement, and/or the like. Similarly, the work vehicle 12 may be configured as any other suitable type of vehicle, such as an agricultural harvester, a self-propelled sprayer, and/or the like.

As shown in FIG. 1, the work vehicle 12 may include a pair of front track assemblies 16, a pair or rear track assemblies 18, and a frame or chassis 20 coupled to and supported by the track assemblies 16, 18. An operator's cab 22 may be supported by a portion of the chassis 20 and may house various input devices for permitting an operator to control the operation of one or more components of the work vehicle 12 and/or one or more components of the implement 10. Additionally, as is generally understood, the work vehicle 12 may include an engine 24 and a transmission 26 mounted on the chassis 20. The transmission 26 may be operably coupled to the engine 24 and may provide variably adjusted gear ratios for transferring engine power to the track assemblies 16, 18 via a drive axle assembly (not shown) (or via axles if multiple drive axles are employed).

As shown in FIGS. 1 and 2, the implement 10 may include a frame 28. More specifically, as shown in FIG. 2, the frame 28 may extend longitudinally between a forward end 30 and an aft end 32. The frame 28 may also extend laterally between a first side 34 and a second side 36. In this respect, the frame 28 generally includes a plurality of structural frame members 38, such as beams, bars, and/or the like, configured to support or couple to a plurality of components. Furthermore, a hitch assembly 40 may be connected to the frame 28 and configured to couple the implement 10 to the work vehicle 12. Additionally, a plurality of wheels 42 (one is shown) may be coupled to the frame 28 to facilitate towing the implement 10 in the direction of travel 14.

In several embodiments, the frame 28 may be configured to support various ground-engaging tools. For instance, the frame 28 may support one or more gangs or sets 44 of disk blades 46. Each disk blade 46 may be configured to penetrate into or otherwise engage the soil as the implement 10 is being pulled through the field. In this regard, the various disk gangs 44 may be oriented at an angle relative to the direction of travel 14 to promote more effective tilling of the soil. In the embodiment shown in FIGS. 1 and 2, the implement 10 includes four disk gangs 44 supported on the frame 28 adjacent to its forward end 30. However, it should be appreciated that, in alternative embodiments, the implement 10 may include any other suitable number of disk gangs 44, such as more or fewer than four disk gangs 44. Furthermore, in one embodiment, the disk gangs 44 may be mounted to the frame 28 at any other suitable location, such as adjacent to its aft end 32.

Moreover, as shown, in one embodiment, the implement frame 28 may be configured to support other ground-engaging tools. For instance, in the illustrated embodiment, the frame 28 is configured to support a plurality of shanks 50 configured to rip or otherwise till the soil as the implement 10 is towed across the field. Furthermore, in the illustrated embodiment, the frame 28 is also configured to support one or more finishing tools, such as a plurality of leveling blades 52 and/or rolling (or crumbler) basket assemblies 54 rotatable relative to the frame 28. However, in other embodiments, any other suitable ground-engaging tools may be coupled to and supported by the implement frame 28, such as a plurality closing disks.

Additionally, in several embodiments, the implement 10 may include a plurality of actuators configured to adjust the positions of the implement 10 and/or various ground-engaging tools coupled thereto. For example, in some embodiments, the implement 10 may include a plurality of disk gang actuators 56 (one is shown in FIG. 2), with each disk gang actuator 56 being configured to move or otherwise adjust the orientation or position of one or more of the disk gang assemblies 44 relative to the implement frame 28. Similarly, in some embodiments, the implement 10 may include a plurality of shank frame actuator(s) 58, with each shank frame actuator 58 being configured to move or otherwise adjust the orientation or position of one or more of the shanks 50 relative to the implement frame 28. Additionally, or alternatively, in some embodiments, the implement 10 may include a plurality of basket frame actuator(s) 59, with each actuator 59 being configured to move or otherwise adjust the orientation or position of a basket frame supporting one or more of the basket assemblies 54 and/or one or more of the leveling blades 52 relative to the implement frame 28.

The implement 10 and/or the work vehicle 12 may additionally be equipped with different types of field condition sensors for monitoring field conditions (e.g., soil moisture) within the field during the performance of an agricultural operation with the implement 10. For instance, one or more field condition sensor(s) 55 may be supported on the vehicle 12 and/or on the implement 10, with each of the sensor(s) 55 being configured to generate data indicative of one or more field conditions, particularly data indicative of a moisture content of the field.

In some instances, the sensor(s) 55 may be non-contact sensors spaced apart from and above a surface of the field during an agricultural operation with the implement 10 while having a field of view generally directed towards a portion of the field. In some embodiments, the field of view of each of the non-contact sensor(s) 55 is directed towards a portion of the field that has yet to be worked by the implement 10, such that there is sufficient time to process the data generated by the non-contact sensor(s) 55 before the implement 10 passes over the detected area. For instance, the field of view of the sensor(s) 55 may be directed in front of the vehicle 12, in front of the implement 10 (e.g., at least in front of the ground-engaging tool(s) being monitored for plugging), and/or towards an adjacent swath to the current swath the implement is currently traveling in that the implement 10 will traverse during a later pass. However, it should be appreciated that, in some embodiments, the non-contact sensor(s) 55 may be mounted on a vehicle configured to perform a separate pass across the field, such as on an unmanned aerial vehicle (UAV) and/or the like, such that the sensor(s) 55 may generate data before and/or during the performance of the agricultural operation with the agricultural implement 10. The non-contact field condition sensor(s) 55 may be any suitable non-contact sensor, such as a ground penetrating radar (GPR) sensor(s), such as a single-frequency GPR sensor or a multi-frequency GPR sensor, a reflectance sensor (e.g., a near-infrared sensor), a camera (e.g., a multispectral camera, an infrared camera, and/or the like), a gamma ray sensor, and/or the like.

In some embodiments, one or more of the sensor(s) 55 is configured as a contact-based sensor, configured to engage the field as the implement 10 performs an operation within the field. In some instances, the contact-based sensor(s) 55 are configured to contact a portion of the field yet to be worked by the implement 10, such that there is sufficient time to process the data generated by the contact-based sensor(s) 55 before the implement 10 passes over the detected area. For instance, the contact-based sensor(s) 55 may be configured to engage the field in front of the vehicle 12, in front of the implement 10 (e.g., at least in front of the ground-engaging tool(s) being monitored for plugging), and/or towards an adjacent swath to the current swath the implement is currently traveling in that the implement 10 will traverse during a later pass. However, it should be appreciated that, in some embodiments, the contact-based sensor(s) 55 may be mounted on a separate vehicle configured to perform a separate pass across the field, such that the contact-based sensor(s) 55 may generate data before and/or during the performance of the agricultural operation with the agricultural implement 10. The contact-based field condition sensor(s) 55 may be any suitable contact-based sensor, such as a capacitance sensor.

As will be described in greater detail below, the data from the field condition sensor(s) 55 may be used to adjust the output frequency of plugging sensors of a system for monitoring plugging of one or more of the basket assemblies 54 of the implement 10 and/or to adjust interpretation of the data generated by such plugging sensors. For instance, the moisture content within the field may affect the degree of attenuation of wireless signals (e.g., ground-penetrating radar signals, LIDAR signals, and/or the like). For example, the higher the moisture content within the field, the higher the degree of attenuation of ground-penetrating radar signals, LIDAR signals, and/or the like. As such, when the moisture content in the field is high, more significant plugging may be detected than is actually occurring, which requires more frequent than necessary inspections of the ground-engaging tools and thus, reduces productivity. Thus, in some instances, an output frequency at which the plugging sensor(s) is configured to transmit the wireless signals and/or baselines/differences for evaluating the return signals may be adjusted based at least in part on the field conditions (e.g., moisture content) within the field before determining whether the ground-engaging tool is experiencing a plugged condition. Therefore, the adjusted output frequency and/or adjusted baselines/differences may be selected to avoid false positives or over estimation of plugging severity when monitoring for a plugging condition of ground-engaging tools, which reduces inspection time and increases productivity.

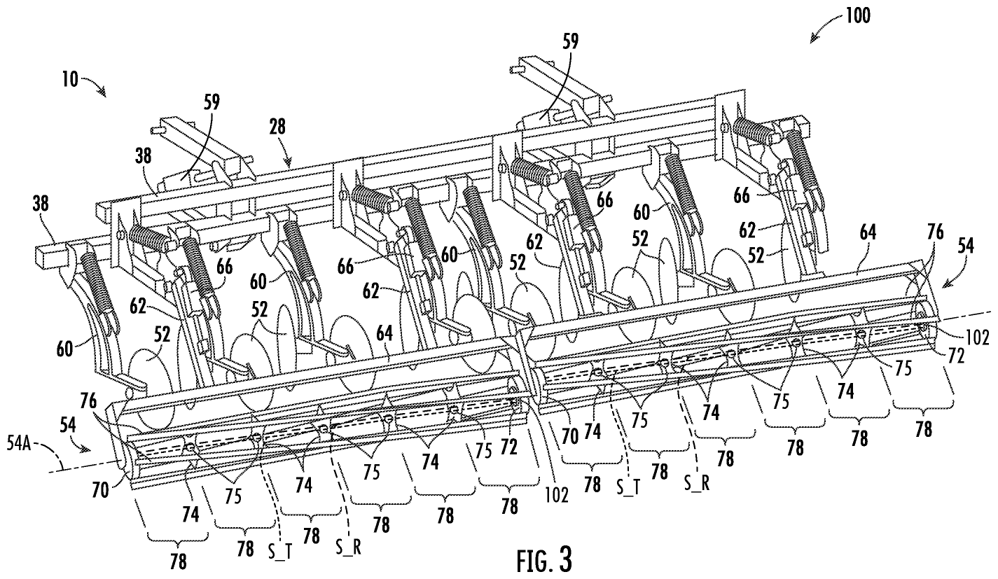

Referring now to FIG. 3, a partial, perspective view of the aft end of the implement 10 shown in FIGS. 1 and 2 is illustrated in accordance with aspects of the present subject matter, particularly illustrating a portion of the finishing tools 52, 54 of the implement 10. As shown, the various finishing tools 52, 54 may be coupled to or supported by the implement frame 28, such as by coupling each tool to a toolbar or laterally extending frame member 38 of the frame 38. For instance, as shown in FIG. 3, a blade support arm 60 may be coupled between a given frame member 38 and each leveling blade 52 or set of leveling blades 52 to support the blades 52 relative to the frame 28. Similarly, one or more basket support arms 62 may be coupled between a given frame member 38 and an associated mounting yoke or basket hanger 64 for supporting each basket assembly 54 relative to the frame 28. Additionally, as shown in FIG. 3, in one embodiment, a basket actuator 66 (e.g., a hydraulic or pneumatic cylinder) may be coupled to each basket support arm 62 to allow the down force or down pressure applied to each basket assembly 54 to be adjusted. The basket actuator(s) 66 may also allow the basket assemblies 54 to be raised off the ground, such as when the implement 10 is making a headland turn and/or when the implement 10 is being operated within its transport mode.

In several embodiments, each basket assembly 54 includes a plurality of support plates 70, 72, 74 configured to support a plurality of blades or bars 76 spaced circumferentially about the outer perimeter of the basket. For instance, as shown in FIG. 3, each basket assembly 54 extends across a lateral width defined between first and second end plates 70, 72 positioned at the opposed lateral ends of the basket assembly 54, spaced apart along a rotational axis 54A of the basket assembly 54. Additionally, each basket assembly 54 includes a plurality of inner support plates 74 spaced apart laterally from one another between the end plates 70, 72. In some instances, each of the inner support plates 74 may include an opening 75, such as a central opening extending therethrough at the rotational axis 54A. Lateral basket sections 78 are generally defined between each pair of adjacent support plates 70, 72, 74, with each basket section 78 being generally characterized by a hollow or substantially hollow interior area surrounded by the lateral portions of the bars 76 extending between the respective pair of adjacent support plates 70, 72, 74. As is generally understood, the end plates 70, 72 may be rotatably coupled to the corresponding basket hanger 64 (which, in turn, is coupled to the associated bracket support arm(s) 62) via bearings to allow the basket assembly 54 to rotate relative to the hanger/arm 64, 62 about the rotational axis 54A as the implement 10 is being moved across the field. Additionally, in the illustrated embodiment, the bars 76 of each basket assembly 54 are configured as formed bars. However, in other embodiments, the bars 76 may have any other suitable configuration, such as flat bars, round bars, and/or the like.

Moreover, in accordance with aspects of the present subject matter, FIG. 3 also illustrates components of one embodiment of a system 100 for monitoring plugging of an agricultural implement. Specifically, in the illustrated embodiment, the system 100 is shown as being configured for use in monitoring for plugged condition(s) of the basket assemblies 54 of the agricultural implement 10. However, in other embodiments, the system 100 may be utilized to identify a plugged condition of any other suitable ground-engaging tool(s).

As shown in FIG. 3, the system 100 includes one or more plugging sensors 102. In general, the plugging sensor(s) 102 are configured to transmit detection signals S_T at multiple, different frequencies across a field of detection and to generate data indicative of return signals S_R received based on reflection of the detection signals S_T at the different frequencies off at least one surface. The plugging sensor(s) 102 may be positioned within an interior of an associated basket assembly 54 such that the field of detection extends generally along the rotational axis 54A. In some embodiments, the plugging sensors 102 are positioned such that the field of detection, and thus, at least a portion of each of the signals S_T. S_R, of the plugging sensor(s) 102 extends through the opening 75 of one or more of the inner support plates 74. For example, in some instances, the plugging sensor(s) 102 are positioned at or on one or both of the end plates 70, 72 of the associated basket assembly 54, with each plugging sensor 102 positioned on one of the end plates 70, 72 directing the field of direction towards the other of the end plates 70, 72 at the other lateral end of the basket assembly 54. In one instance, the plugging sensor(s) 102 are positioned on the rotational axis 54A. For instance, the plugging sensor(s) 102 may be positioned at a rotational bearing(s) (not shown) of the associated basket assembly 54 at the end plate(s) 70, 72, which allow rotation of the basket assembly 54 relative to the implement frame 28, such that the field of detection extends along the rotational axis 54A. However, it should be appreciated that the plugging sensor(s) 102 may be coupled to or supported by any other suitable component(s) of the basket assembly 54, such as within the opening(s) 75 of the support plate(s) 74 and or any other suitable location. While the plugging sensors 102 are only shown as being supported on the end plates 72, in some instances, depending on the strength of the plugging sensor(s) 102, one of the plugging sensors 102 may be positioned at or on each of the end plates 70, 72 of a respective basket assembly 54 such that the signal strength may be enough to cover the entire lateral width of the basket assembly 54 when the basket assembly 54 is plugged. It should additionally be appreciated that, in some embodiments, at least one plugging sensor 102 is positioned within each basket assembly 54 of the implement 10 such that the plugging condition of each basket assembly 54 may be individually monitored.

The plugging sensor(s) 102 may be any suitable multi-frequency sensor, such as a multi-frequency GPR sensor, a multi-frequency radar sensor, a multi-frequency LIDAR device, and/or the like. It should be appreciated that, depending on the sensor type of the plugging sensor(s), in some embodiments, the signals S_T transmitted by the plugging sensor(s) 102 may at least partially transmit through field materials, but less so, or not at all, through the material of the support plates 74. Generally, the signals S_T transmitted will attenuate at the support plates 74 and experience phase shift and/or attenuate when moving through accumulated field materials, such that the return signals S_R are changed in strength and/or phase from the corresponding transmitted signals S_T.

The different frequencies for the signals S_T transmitted by the plugging sensor(s) 102 may be associated with different distances along the lateral width of the basket assembly 54. For instance, detection signals S_T of the plugging sensor(s) 102 transmitted with higher frequencies may travel a shorter distance than detection signals S_T of the plugging sensor(s) 102 transmitted with lower frequencies. As such, in some instances, the lower frequencies of the plugging sensor(s) 102 may generally be used to at least monitor lateral basket sections 78 of the basket assembly 54 further away from the originating plugging sensor 102 than the higher frequencies of the plugging sensor(s) 102.

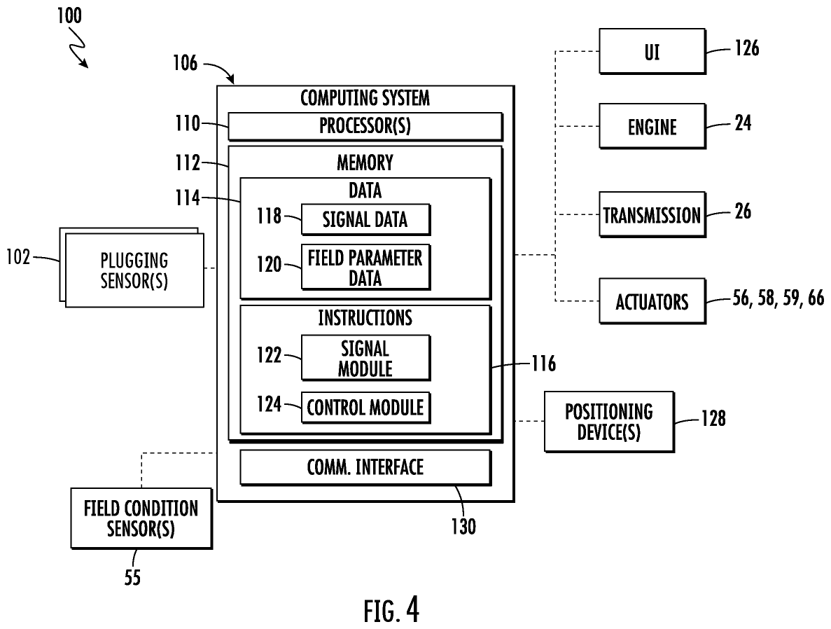

Moreover, the return signals S_R detected by the plugging sensor(s) 102 for each transmitted signal frequency S_T may have a respective baseline or expected return signal profile when the basket assembly 54 is not experiencing the plugged condition (is unplugged). For instance, as the basket assembly 54 becomes plugged, a return signal profile of the return signal(s) S_R reflected from one or more of the different frequencies may change (e.g., from the respective baseline return signal profile). For instance, the return signals S_R may attenuate more quickly or indicate a shorter reflection distance when the basket assembly 54 starts plugging than when the basket assembly 54 is unplugged. As such, as will be described below, an associated computing system 202 (FIG. 4) of the disclosed system 100 may be configured to infer or determine when the associated basket assembly 54 is likely experiencing the plugged condition based at least in part on the data generated by the plugging sensor(s) 102. Once it is determined that the basket assembly 54 is experiencing a plugged condition, an appropriate control action may then be executed, such as by notifying the operator of the plugged condition or by performing an automated adjustment of an operation of the implement 10 and/or the associated work vehicle 12.

Referring now to FIG. 4, a schematic view of one embodiment of a system 100 for monitoring plugging of basket assemblies of an agricultural implement is illustrated in accordance with aspects of the present subject matter. In general, the system 100 will be described with reference to the implement shown in FIGS. 1 and 2 and the basket assembly 54 and associated system components shown in FIG. 3. However, in other embodiments, the disclosed system 100 may be utilized to monitor plugging of ground-engaging tools of any other suitable agricultural implement having any other suitable implement configuration and/or with any other suitable ground-engaging tool(s) having any other suitable tool configuration. Additionally, it should be appreciated that, for purposes of illustration, communicative links or electrical couplings of the system 100 shown in FIG. 4 are indicated by dashed lines.

As indicated above, in several embodiments, the system 100 may include a computing system 106 and various other components configured to be communicatively coupled to and/or controlled by the computing system 106, such as the plugging sensor(s) 102, field condition sensors (e.g., the field condition sensor(s) 55), drive components of the work vehicle 12 (e.g., the engine 24, the transmission 26, and/or the like), one or more actuator(s) of the implement 10 (e.g., the disk gang actuator(s) 56, the shank frame actuator(s) 58, the basket frame actuator(s) 59, the basket actuator(s) 66, and/or the like), and/or any other suitable components. Moreover, in some instances, the system 100 may include a user interface (e.g., user interface(s) 126). The user interface(s) 126 may include, without limitation, any combination of input and/or output devices that allow an operator to provide operator inputs to the computing system 106 and/or that allow the computing system 106 to provide feedback to the operator, such as a keyboard, keypad, pointing device, buttons, knobs, touch sensitive screen, mobile device, audio input device, audio output device, and/or the like. Additionally, in some instances, the system 100 may include one or more positioning devices communicatively coupled to the computing system 106 and configured to generate data indicative of the location of the agricultural implement 10 and/or vehicle 12, such as a satellite navigation positioning device (e.g., a GPS system, a Galileo positioning system, a Global Navigation satellite system (GLONASS), a BeiDou Satellite Navigation and Positioning system, a dead reckoning device, and/or the like).

In general, the computing system 106 may correspond to any suitable processor-based device(s), such as a computing device or any combination of computing devices. Thus, as shown in FIG. 4, the computing system 106 may generally include one or more processor(s) 110 and associated memory devices 112 configured to perform a variety of computer-implemented functions (e.g., performing the methods, steps, algorithms, calculations, and the like disclosed herein). As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory 112 may generally comprise memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory 112 may generally be configured to store information accessible to the processor(s) 110, including data 114 that can be retrieved, manipulated, created and/or stored by the processor(s) 110 and instructions 116 that can be executed by the processor(s) 110.

It should be appreciated that the computing system 106 may correspond to an existing controller for the implement 10 and/or the vehicle 12 or may correspond to a separate processing device. For instance, in one embodiment, the computing system 106 may form all or part of a separate plug-in module that may be installed in operative association with the implement 10 and/or the vehicle 12 to allow for the disclosed system and method to be implemented without requiring additional software to be uploaded onto existing control devices of the implement 10 and/or the vehicle 12.

In several embodiments, the data 114 may be stored in one or more databases. For example, the memory 112 may include a signal database 118 for storing the data generated by the plugging sensor(s) 102 and/or data associated with such data generated by the plugging sensor(s) 102. Specifically, in one embodiment, the data generated by the plugging sensor(s) 102 and stored within the signal database 118 associates different frequencies of the detection signals S_T transmitted with corresponding return signals S_R detected or received by the plugging sensor(s) 102 based on reflection of the detection signals S_T transmitted at the different frequencies off at least one surface (e.g., the support plates 74 and/or field materials (if present)) within the monitored basket assembly(ies) 54). As will be described below, the return signals S_R may be indicative of a return signal profile across the lateral width of the basket assembly 54, which may be compared to stored baseline return signal profiles generated when the monitored basket assembly(ies) 54 were known to be unplugged, to determine when the monitored basket assembly(ies) 54 are experiencing a plugged condition. The data stored in the signal database 118 may be cross-referenced with position data from the positioning device(s) 128 such that locations in the field associated with plugging may be accounted for in subsequent passes of the agricultural implement 10 and/or in subsequent agricultural operations (e.g., planting) within the field.

Additionally, as shown in FIG. 4, the memory 112 may include a field parameter database 120 for storing information related to one or more parameters of the field being processed during the performance of the associated agricultural operation (e.g., a tillage operation). For instance, in one embodiment, moisture data associated with the moisture content or level of the soil within the field may be stored within the field parameter database 120. As indicated above, the wetness or moisture content of the soil may impact the magnitude of the signal degradation or attenuation of the wireless signals being transmitted through adjacent material accumulation and/or induce a phase shift on the wireless signals associated with such material accumulation. For instance, material accumulation including significantly wet soil may attenuate the signals S_T transmitted from and/or return signals S_R detected by an adjacent plugging sensor 102 to a greater degree than material accumulation including drier or less wet soil. Accordingly, by knowing the soil moisture within the field, the computing system 106 may be configured to more accurately assess the signals S_R received by the plugging sensor(s) 102.

It should be appreciated that the moisture data may be correspond to pre-existing or predetermined moisture data stored within the field parameter database 120 or the moisture data may correspond to sensor data that is being actively collected or generated during the performance of the associated agricultural operation. For instance, in one embodiment, the computing system 106 may be provided with soil moisture data (e.g., in the form of a soil moisture map) that was collected during a previous agricultural operation or that was generated based on previously known data associated with the field conditions. Alternatively, or additionally, the moisture data may be the data generated by the field condition sensor(s) 55 supported on the implement 10 and/or vehicle 12, and/or from any other suitable moisture sensor(s). The data from the field condition sensor(s) 55 may be cross-referenced with position data from the positioning device(s) 128 such that the moisture content at different locations in the field may be mapped. In some instances, the data from the field condition sensor(s) 55 is cross-referenced to the soil type at the location in the field to more accurately determine the moisture content at the location in the field from the moisture data generated by the sensor(s) 55.

Referring still to FIG. 4, in several embodiments, the instructions 116 stored within the memory 112 of the computing system 106 may be executed by the processor(s) 110 to implement a signal module 122. In general, the signal module 122 may be configured to control the plugging sensor(s) 102 to transmit the wireless signals S_T at a plurality of output frequencies, where each output frequency is associated with a different distances along the lateral width of the associated basket assembly(ies) 54. In some instances, the signal module 122 may be configured to select the output frequency(ies) at which wireless signals S_T are to be transmitted from the plugging sensor(s) 102 e.g., based at least in part on the moisture content of the field.

For example, in several embodiments, the signal module 122 may determine the moisture content within the field based at least in part on moisture data (e.g., from the field parameter database 120), then determine an adjusted or corresponding set of output frequencies at which the plugging sensor(s) 102 transmits the wireless signals S_T based at least in part on the moisture content within the field. Generally, to account for increased attenuation for higher moisture contents of the field, higher output frequencies are selected. The signal module 122 may determine the adjusted output frequencies using a look-up table, an algorithm, and/or the like stored, for example, in the memory 112, that correlates the moisture content within the field to a range of output frequencies for the transmission of the wireless signals S_T. In some instances, the corresponding range of output frequencies for the moisture content is selected such that the reflected wireless signals S_R will not fully attenuate, or will only fully attenuate, when the associated ground-engaging tools experience severe plugging conditions. The signal module 122 may, in some embodiments, compare the current output frequencies of the plugging sensor(s) 102 to the adjusted output frequencies, then adjust the output frequencies from the current output frequencies to the adjusted output frequencies when the current output frequencies differ from the adjusted output frequencies. In some instances, the signal module 122 may only adjust the output frequencies from the current output frequencies to the adjusted output frequencies when the current output frequencies differ from the adjusted output frequencies by at least a threshold amount.

Additionally, or alternatively, the signal module 122 may be configured to compare the return wireless signals S_R detected at the plugging sensor(s) 102 to one or more associated return profiles. For instance, in one embodiment, the signal module 122 may compare each of the wireless signals S_R detected at the plugging sensor(s) 102 in response to the signals S_T transmitted at different frequencies by the plugging sensor(s) 102 to each respective baseline return signal profile associated with the different frequencies to determine when the basket assembly(ies) 54 are experiencing the plugged condition. For instance, in general, when the basket assemblies 54 are not experiencing the plugged condition (are unplugged), the transmitted signals S_T at the different frequencies typically only attenuate at the individual support plates 74, and the return signals S_R detected at the plugging sensor(s) 102 in response to the signals S_T transmitted at the different frequencies by the plugging sensor(s) 102 may substantially match the strength (attenuation pattern of the support plates 74) and phase of the respective baseline return signal profiles associated with the different frequencies. Conversely, when the basket assemblies 54 are experiencing the plugged condition, the transmitted signals S_T attenuate at the individual support plates 74 in addition to becoming phase shifted and further attenuated as they move through the accumulated plugging materials (e.g., field materials), such that the return signals S_R detected at the plugging sensor(s) 102 in response to the signals S_T transmitted by the plugging sensor(s) 102 at different frequencies begin to deviate from one or more of the respective baseline return signal profiles associated with the different frequencies. As such, the computing system 106 may determine when the monitored basket assembly(ies) 54 are experiencing the plugged condition based on a difference in strength and/or phase between the return signals S_R and the associated baseline return profiles.

Once the return signal S_R for one or more frequencies (and thus, one or more lateral basket sections 78) differs in signal strength (attenuates) from the associated baseline return profile by at least a given amount and/or is phase shifted by at least a given amount from the associated baseline return profile, the computing system 106 may then infer or estimate that the associated basket assembly 54 is currently experiencing a plugged condition and may initiate appropriate control actions in response to the detection of the plugged condition. It should be appreciated that the difference in signal strength and/or difference in phase may be selected based at least in part on the moisture content within the field (e.g., based at least in part on the soil moisture data stored within the field parameter database 120). For instance, when the moisture content within the field is higher, the difference in signal strength and/or difference in phase may be selected by the signal module 122 to be higher than when the moisture content is lower. In one example, a look-up table may be stored within the memory 112 that correlates soil moisture values to corresponding differences associated with the degree to which each moisture level degrades (attenuates) or phase shifts the wireless signals.

Additionally, the computing system 106 may be configured to identify the severity of any detected plugged condition. For instance, the computing system 106 may be configured to identify the severity of any detected plugged condition based on the magnitude of the attenuations in the signal strength, the magnitude of the phase shifts, the number of return signals S_R that differ by the given amount(s) in signal strength, and/or the number of return signals S_R that phase shift from the respective baseline return signal profiles. For instance, there may be multiple plugging-related difference thresholds (e.g., a lower magnitude attenuation/phase shift for a partially plugged condition, and a higher magnitude attenuation/phase shift for a fully plugged condition), and/or multiple count thresholds (e.g., a lower count of return signals S_R that differ from the baseline return signal profiles for a partially plugged condition, and a higher count of return signals S_R that differ from the baselines return profiles for a fully plugged condition). The computing system 106 may infer or estimate the severity of the plugged condition by comparing each of the return signals S_R to each of such thresholds, which may impact the selection of the appropriate control action(s) to be executed (e.g., notifying the operator when it is detected that the tool is partially plugged versus performing an automated control action to adjust the operation of the implement when it is detected that the tool is fully plugged).

The signal module 122 may further be configured to correlate the data indicative of the return signals S_R with the plugging sensor 102 generating such data, such as by identifying a unique code or number (e.g., a serial number) transmitted from each plugging sensor 102. By doing so, the signal module 122 may be configured to not only assess the signal strength/phase of the return signals S_R received from the plugging sensor(s), but also determine which basket assembly(ies) 54 is plugging.

Referring still to FIG. 4, the instructions 116 stored within the memory 112 of the computing system 106 may also be executed by the processor(s) 110 to implement a control module 124. In general, the control module 124 may be configured to initiate a control action when it is determined that at least one of the basket assemblies 54 of the implement 10 is experiencing a plugged condition. As indicated above, in one embodiment, the control module 124 may be configured to provide a notification to the operator of the vehicle 12 and/or implement 10 indicating that material accumulation is present on, within, and/or adjacent to the basket assembly(ies) 54 of the implement 10. For instance, in one embodiment, the control module 124 may control an operation of the user interface 126 to indicate (e.g., cause a visual or audible notification or indicator to be presented) to an operator that the basket assembly(ies) 54 is plugged, recommend an action to mitigate the plugging, and/or the like.

In one embodiment, the control module 124 may be configured to execute an automated control action designed to adjust the operation of the implement 10 and/or the vehicle 12. For instance, in one embodiment, the computing system 106 may be configured to increase or decrease the operational or ground speed of the implement 10 in an attempt to reduce the amount of material accumulation and/or to limit further material accumulation. For instance, as shown in FIG. 4, the computing system 106 may be communicatively coupled to both the engine 24 and the transmission 26 of the work vehicle 12. In such an embodiment, the computing system 106 may be configured to adjust the operation of the engine 24 and/or the transmission 26 in a manner that increases or decreases the ground speed of the work vehicle 12 and, thus, the ground speed of the implement 10, such as by transmitting suitable control signals for controlling an engine or speed governor (not shown) associated with the engine 24 and/or transmitting suitable control signals for controlling the engagement/disengagement of one or more clutches (not shown) provided in operative association with the transmission 26. It should be appreciated that computing system 106 may also be configured to decrease the ground speed in a manner that brings the implement 10 and the vehicle 12 to a complete stop.

In addition to the adjusting the ground speed of the implement 10 and the vehicle 12 (or as an alternative thereto), the computing system 106 may be configured to adjust an operating parameter associated with the basket assembly(ies) 54 of the implement 10. For instance, as shown in FIG. 4, the computing system 106 may be configured to control the operation of one or more actuators 59, 66 (e.g., control valves, pumps, and/or the like associated with the actuators 59, 66) of the implement 10 to automatically adjust the penetration depth, the down force, and/or any other suitable operating parameter associated with the basket assembly(ies) 54 of the implement 10 to mitigate plugging. For instance, by controlling the operation of the basket actuators 66, the computing system 106 may automatically adjust the down force or down pressure applied to the associated basket assembly 54 (e.g., cyclically increase and decrease engagement with the field), which may help mitigate plugging. Additionally, or alternatively, the computing system 106 may control the operation of the basket actuator 66 to raise and lower the associated basket assembly 54 relative to the ground to indicate to an operator that the basket assembly 54 needs to be de-plugged.

Moreover, as shown in FIG. 4, the computing system 106 may also include a communications interface 130 to provide a means for the computing system 106 to communicate with any of the various other system components described herein. For instance, one or more communicative links or interfaces (e.g., one or more data buses) may be provided between the communications interface 130 and the plugging sensor(s) 102 to allow the computing system 106 to control the plugging sensor(s) 102 output frequency for transmitting the output signals S_T and/or to receive the return signals S_R (and/or related signal data) received by the plugging sensor(s) 102. Similarly, one or more communicative links or interfaces (e.g., one or more data buses) may be provided between the communications interface 130 and the engine 24, the transmission 26, the user interface 126, the actuator(s) 56, 58, 59, 66, and/or the like to allow the computing system 106 to control the operation of and/or otherwise communicate with such system components. Moreover, one or more communicative links or interfaces (e.g., one or more data buses) may be provided between the communications interface 130 and the field condition sensor(s) 55 to allow the computing system 106 to control the field condition sensor(s) 55 to collect field condition data and/or to receive the field condition data from the field condition sensor(s). Additionally, one or more communicative links or interfaces (e.g., one or more data buses) may be provided between the communications interface 130 and the positioning device(s) 128 to allow the computing system 106 to control the positioning device(s) 128 to collect the position data and/or to receive the position data from the positioning device(s) 128.

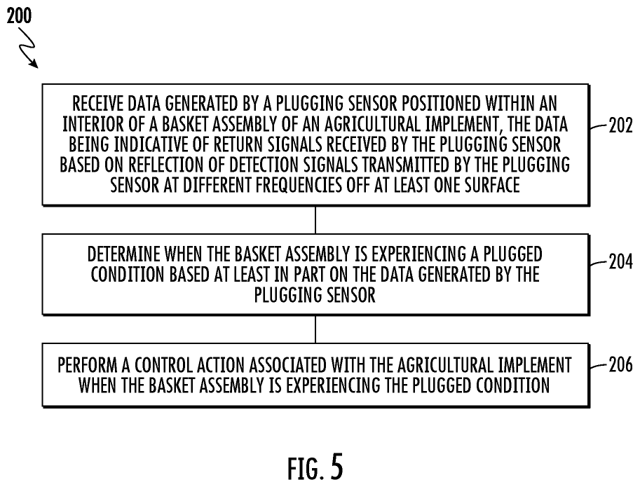

Referring now to FIG. 5, a flow diagram of one embodiment of a method 200 for monitoring plugging of basket assemblies of an agricultural implement is illustrated in accordance with aspects of the present subject matter. In general, the method 200 will be described herein with reference to the agricultural implement 10, the basket assembly 54, and the system 100 described above with reference to FIGS. 1-4. However, it should be appreciated by those of ordinary skill in the art that the disclosed method 200 may generally be implemented with any agricultural implement having any suitable implement configuration, any ground-engaging tool having any suitable tool configuration, and/or any system having any suitable system configuration. In addition, although FIG. 5 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As shown in FIG. 5, at (202), the method 200 may include receiving data generated by a plugging sensor positioned within an interior of the basket assembly of an agricultural implement, the data being indicative of return signals received by the plugging sensor based on reflection of detection signals transmitted by the plugging sensor at different frequencies off at least one surface. For instance, as described above, the computing system 106 may be configured to receive data generated by the plugging sensor(s) 102 positioned within an interior of an associated basket assembly 54 of the agricultural implement 10, with the data being indicative of the return signals S_R received by the plugging sensor(s) 102 based on reflection off at least one surface of the detection signals S_T transmitted by the plugging sensor(s) 102 at multiple, different frequencies.

Moreover, at (204), the method 200 may include determining when the basket assembly is experiencing a plugged condition based at least in part on the data generated by the plugging sensor. For example, as discussed above, the computing system 106 may determine that the monitored basket assembly 54 is experiencing a plugged condition based at least in part on the data generated by the plugging sensor(s) 102. For instance, when the return signals S_R for one or more frequencies of the transmitted signals S_T are determined to differ by at least a given difference from a baseline return signal profile, the computing system 106 may determine that the basket assembly 54 is experiencing the plugged condition.

Additionally, at (206), the method 200 may include performing a control action associated with the agricultural implement when the basket assembly is experiencing the plugged condition. For example, as described above, the computing system 106 may automatically control an operation of the user interface(s) 126, the engine 24, the transmission 26, the actuator(s) 59, 66, and/or the like when the monitored basket assembly 54 is experiencing the plugged condition and/or a plugged condition of a determine threshold/magnitude.

It is to be understood that the steps of the method 200 are performed by the computing system 106 upon loading and executing software code or instructions which are tangibly stored on a tangible computer readable medium, such as on a magnetic medium, e.g., a computer hard drive, an optical medium, e.g., an optical disk, solid-state memory, e.g., flash memory, or other storage media known in the art. Thus, any of the functionality performed by the computing system 106 described herein, such as the method 200, is implemented in software code or instructions which are tangibly stored on a tangible computer readable medium. The computing system 106 loads the software code or instructions via a direct interface with the computer readable medium or via a wired and/or wireless network. Upon loading and executing such software code or instructions by the computing system 106, the computing system 106 may perform any of the functionality of the computing system 106 described herein, including any steps of the method 200 described herein.

The term “software code” or “code” used herein refers to any instructions or set of instructions that influence the operation of a computer or computing system. They may exist in a computer-executable form, such as machine code, which is the set of instructions and data directly executed by a computer's central processing unit or by a computing system, a human-understandable form, such as source code, which may be compiled in order to be executed by a computer's central processing unit or by a computing system, or an intermediate form, such as object code, which is produced by a compiler. As used herein, the term “software code” or “code” also includes any human-understandable computer instructions or set of instructions, e.g., a script, that may be executed on the fly with the aid of an interpreter executed by a computer's central processing unit or by a computing system.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. An agricultural system for monitoring plugging of basket assemblies of an agricultural implement, the agricultural system comprising:
a basket assembly configured to be supported by a frame of an agricultural implement for rotation relative to the frame about a rotational axis, the basket assembly extending across a lateral width defined between a first lateral end and a second lateral end spaced apart along the rotational axis;
a plugging sensor positioned within an interior of the basket assembly such that the plugging sensor is configured to transmit detection signals at different frequencies across a field of detection along the interior of the basket assembly and along the rotational axis, the plugging sensor being configured to generate data indicative of return signals received based on reflection of the detection signals at the different frequencies off at least one surface; and
a computing system configured to:
receive the data generated by the plugging sensor; and
determine when the basket assembly is experiencing a plugged condition based at least in part on the data generated by the plugging sensor.

2. The agricultural system of claim 1, wherein the plugging sensor is positioned at one of the first and second lateral ends and directs the field of detection towards another of the first and second lateral ends.

3. The agricultural system of claim 1, wherein the basket assembly comprises:
a plurality of support plates spaced out along the rotational axis, each of the plurality of support plates defining an opening extending therethrough at the rotational axis; and
a plurality of blades supported by the plurality of support plates such that blades of the plurality of blades are spaced apart from each other about the rotational axis,
wherein the plugging sensor directs the field of detection through a plurality of the openings defined in the plurality of support plates.

4. The agricultural system of claim 1, wherein each of the different frequencies is associated with a respective baseline return signal profile when the basket assembly is not experiencing the plugged condition, and
wherein the computing system is configured to determine when the basket assembly is experiencing the plugged condition based on a difference between each of the return signals associated with the different frequencies from the data generated by the plugging sensor and the respective baseline return signal profile.

5. The agricultural system of claim 4, wherein the difference includes at least one of attenuation or phase shift.

6. The agricultural system of claim 4, wherein each of the different frequencies is also associated with a different distance along the lateral width of the basket assembly when the basket assembly is not experiencing the plugged condition.

7. The agricultural system of claim 1, wherein the computing system is further configured to perform a control action in response to determining that the basket assembly is experiencing the plugged condition.

8. The agricultural system of claim 1, wherein the computing system is further configured to control the plugging sensor to transmit the detection signals at the different frequencies.

9. The agricultural system of claim 1, wherein the plugging sensor is at least one of a multi-frequency ground penetrating radar sensor, a multi-frequency radar sensor, or a LIDAR device.

10. The agricultural system of claim 1, further comprising a soil moisture sensor configured to generate moisture data indicative of soil moisture within a field during an agricultural operation with the agricultural implement,
wherein the computing system is further configured to receive the moisture data, and
wherein the computing system is configured to determine when the basket assembly is experiencing the plugged condition based at least in part on the data generated by the plugging sensor and the moisture data.

11. An agricultural method for monitoring plugging of a basket assembly of an agricultural implement, the basket assembly being configured to be supported by a frame of the agricultural implement for rotation relative to the frame about a rotational axis, the basket assembly extending across a lateral width defined between a first lateral end and a second lateral end spaced apart along the rotational axis, the agricultural method comprising:
receiving, with a computing system, data generated by a plugging sensor positioned within an interior of the basket assembly, the plugging sensor being configured to transmit detection signals at different frequencies across a field of detection along the interior of the basket assembly and along the rotational axis, the data being indicative of return signals received by the plugging sensor based on reflection of the detection signals at the different frequencies off at least one surface;
determining, with the computing system, when the basket assembly is experiencing a plugged condition based at least in part on the data generated by the plugging sensor; and
performing, with the computing system, a control action associated with the agricultural implement when the basket assembly is experiencing the plugged condition.

12. The agricultural method of claim 11, wherein the basket assembly comprises:
a plurality of support plates spaced out along the rotational axis, each of the plurality of support plates defining an opening extending therethrough at the rotational axis; and
a plurality of blades supported by the plurality of support plates such that blades of the plurality of blades are spaced apart from each other about the rotational axis,
wherein receiving the data generated by the plugging sensor comprises receiving the data generated by the plugging sensor indicative of the return signals received by the plugging sensor based on the reflection of the detection signals directed at the different frequencies through a plurality of the openings defined in the plurality of support plates.

13. The agricultural method of claim 11, wherein each of the different frequencies is associated with a respective baseline return signal profile when the basket assembly is not experiencing the plugged condition, and
wherein determining when the basket assembly is experiencing the plugged condition comprises determining when the basket assembly is experiencing the plugged condition based on a difference between each of the return signals at the different frequencies from the data generated by the plugging sensor and the respective baseline return signal profile.

14. The agricultural method of claim 13, wherein the difference includes at least one of attenuation or phase shift.

15. The agricultural method of claim 13, wherein each of the different frequencies is also associated with a different distance along the lateral width of the basket assembly when the basket assembly is not experiencing the plugged condition.

16. The agricultural method of claim 11, wherein performing the control action comprises controlling an operation of the agricultural implement to adjust an operating parameter associated with the agricultural implement when it is identified that the basket assembly is experiencing the plugged condition.

17. The agricultural method of claim 11, wherein performing the control action comprises controlling an operation of a user interface associated with the agricultural implement to indicate the plugging condition of the basket assembly when it is identified that the basket assembly is experiencing the plugged condition.

18. The agricultural method of claim 11, further comprising controlling, with the computing system, the plugging sensor to transmit the detection signals at the different frequencies.

19. The agricultural method of claim 11, wherein receiving the data generated by the plugging sensor comprises receiving the data generated by at least one of a multi-frequency ground penetrating radar sensor, a multi-frequency radar sensor, or a LIDAR device.

20. The agricultural method of claim 11, further comprising receiving, with the computing system, moisture data generated by a soil moisture sensor and indicative of soil moisture within a field during an agricultural operation with the agricultural implement,
wherein determining when the basket assembly is experiencing the plugged condition comprises determining when the basket assembly is experiencing the plugged condition based at least in part on the data generated by the plugging sensor and the moisture data.
